(19) United States
(12) Patent Application Publication
(10) Pub. No.: US 2008/0051382 A1
(43) Pub. Date: Feb. 28, 2008

(54) Crystalline Forms of 3-Beta-Amino 17-Methylene Androstane-6-Alpha 7-Beta-Diol Hydrochloride

(75) Inventors: Colette Colladant, Rosny-Sous-Bois (FR); Denis Prat, Pantin (FR); Pascal Billot, Montreuil (FR); Alexandre Giuliani, Boissy Santi Leger (FR); Hagit Elnaleh, Ormesson sur Marne (FR); Marc-Antoine Perrin, Jouy en Josas (FR)

Correspondence Address: SEED INTELLECTUAL PROPERTY LAW GROUP PLLC, 701 FIFTH AVE, SUITE 5400, SEATTLE, WA, 98104, US

(73) Assignee: INFLAZYME PHARMACEUTICALS LTD., RICHMOND (CA)

(21) Appl. No.: 10/575,689

(22) PCT Filed: Oct. 20, 2004
(86) PCT No.: PCT/IB04/03426

§ 371 Date: May 21, 2007

Related U.S. Application Data
(60) Provisional application No. 60/512,679, filed on Oct. 20, 2003.

(30) Foreign Application Priority Data
Oct. 20, 2003 (FR) 0312257

(51) Int. Cl. A61K 31/565; A61P 29/00; C07J 53/00

(52) U.S. Cl. 514/182; 552/521

(57) ABSTRACT

The invention relates to novel crystalline forms of 3-beta-amino-17-methylene-androstane-6-alpha,7-beta-diol hydrochloride, referred to as form A, form B and form C, a method for preparing same, the use thereof as a drug, and pharmaceutical compositions containing same.

The present invention relates to the discovery of three crystalline forms of 3-beta-amino-17-methylene-androstane-6-alpha,7-beta-diol hydrochloride (compound of formula I), represented by structure:

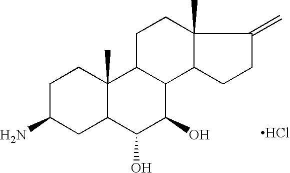

Patent application WO0183512 describes 3-beta-amino-17-methylene-androstane-6-alpha,7-beta-diol, and pharmaceutically acceptable salts thereof for the treatment of inflammatory diseases and, especially, asthma.

Compound 3-beta-amino-17-methylene-androstane-6-alpha,7-beta-diol as described and prepared in WO0183512, is in the form of an acetate salt. This salt in acetate form is hygroscopic, which is a major inconvenience for industrial development.

This invention is directed to one or more new crystalline forms that do not present the inconveniences of the form previously described.

Solid forms, and particularly pharmaceutical products, can present more than one crystalline form. This is referred to as polymorphism. By polymorphous form one means all asolvated forms of a crystallized molecule and by pseudo-polymorphous all solvated forms.

Polymorphous and pseudo-polymorphous forms of the same molecule generally show different physical properties such as solubility, hygroscopicity and stability. It should be noted that at present there is no method that permits with certainty to recognize (experimental triage) or predict (theoretical triage by molecular modeling) the existence of such-and-such a polymorph or pseudo-polymorph , or to predict their physical properties.

Obtaining new polymorphous or pseudo-polymorphous forms of molecules having therapeutic activity holds a major interest for the pharmaceutical industry, especially from the viewpoint of their preparation on an industrial scale, their implementation within pharmaceutical compositions, the search for better stability.

The Applicant has discovered three new crystalline forms of 3-beta-amino-17-methylene-androstane-6-alpha,7-beta-diol hydrochloride (form A, form B and form C). Form A is anhydrous, form B is di-hydrated, and form C is mono-hydrated. In addition to the advantages stated above, crystalline form A shows an absence of hygroscopicity.

In a first embodiment, the invention provides a new crystalline form of anhydrous 3-beta-amino-17-methylene-androstane-6-alpha,7-beta-diol hydrochloride herein defined as form A. The crystalline form A of 3-beta-amino-17-methylene-androstane-6-alpha,7-beta-diol hydrochloride, according to the invention, is in the form of a crystalline powder that is stable from 0 to 90% Relative Humidity (RH) and begins to deteriorate chemically around 240° C., decomposing completely above 280° C. It has been defined below by the indexing of its powder X-ray diffraction pattern diagram.

In another embodiment, the invention provides a new crystalline form of hydrated 3-beta-amino-17-methylene-androstane-6-alpha,7-beta-diol hydrochloride that is hereindefined as form B. It can be used as an intermediate for preparing form A. It is a di-hydrated form that is stable above 50% RH. It is also defined below by the indexing of its powder X-ray diffraction pattern diagram.

In a further embodiment, the invention provides a new crystalline form of hydrated 3-beta-amino-17-methylene-androstane-6-alpha,7-beta-diol hydrochloride that is herein defined as form C. Form C appeared in a mixture with other forms (anhydrous forms D and E). Form C was obtained pure following an additional treatment consisting in maintaining the mixture of forms in an controlled atmosphere with 97% relative humidity for a few days. It is a mono-hydrated form that is stable from 0 to 90% RH. It turns into anhydride D by heating above 60° C. It is also defined below by the indexing of its powder X-ray diffraction pattern diagram.

The crystalline forms A, B or C of the compound of formula I present similar therapeutic activities as those described for the compound 3-beta-amino-17-methylene-androstane-6-alpha,7-beta-diol in WO0183512 application.

They are especially useful in the treatment of inflammatory diseases, and of asthma.

### Powder X-Ray Diffraction

The analyses were carried out on a Philips X'pert Pro X-ray diffractometer having a copper anticathode tube equipped with a front monochromator (wavelength of the copper Ka₁ line: 1.54060 Å). The arrangement is of the Bragg-Brentano type with a Philips X'celerator detector. The angular swept range extends from 2 to 40 degrees 2θ with a step of 0.02 degrees 2θ. The counting period was 300 seconds per step.

### Form A

Form A crystallizes in the monoclinic system (space group P2₁, Z=2), with unit cell parameters at T=295 K as follows:

| | |
|---|---|
| a = 16.058(2) Å, | β = 90.24(2)° |
| b = 6.995(1) Å, | V = 1012.2 Å³ |
| c = 9.011(2) Å | density = 1.168 |

The asymmetric unit is composed of one molecule of 3-beta-amino-17-methylene-androstane-6-alpha,7-beta-diol hydrochloride.

As all lines present on the diffraction diagram are indexed, form A, as obtained according to the crystallization process described below in Example 1 or Example 2, is a pure physical form.

The indexing of the initial 30 lines of the powder X-ray diffraction pattern diagram of form A of 3-beta-amino-17-methylene-androstane-6-alpha,7-beta-diol hydrochloride at T=295 K, in lattice spacing and in “mean λ_{Cu Ka}” 2θ positions, gives the following result:

| | | | | 2-theta |
|---|---|---|---|---|
| | | | Lattice | “mean λCu Kα” |
| h | k | l | spacing (Å) | 1.54184 Å |
| 1 | 0 | 0 | 16.058 | 5.50 |
| 0 | 0 | 1 | 9.011 | 9.82 |
| 2 | 0 | 0 | 8.029 | 11.02 |
| -1 | 0 | 1 | 7.872 | 11.24 |
| 1 | 0 | 1 | 7.844 | 11.28 |
| 1 | 1 | 0 | 6.413 | 13.81 |
| -2 | 0 | 1 | 6.007 | 14.75 |
| 2 | 0 | 1 | 5.982 | 14.81 |
| 0 | 1 | 1 | 5.526 | 16.04 |
| 3 | 0 | 0 | 5.353 | 16.56 |
| 2 | 1 | 0 | 5.274 | 16.81 |
| -1 | 1 | 1 | 5.229 | 16.96 |
| 1 | 1 | 1 | 5.221 | 16.98 |
| 3 | 0 | 1 | 4.610 | 19.25 |
| 3 | 0 | 1 | 4.594 | 19.32 |
| -2 | 1 | 1 | 4.557 | 19.48 |
| 2 | 1 | 1 | 4.546 | 19.53 |
| 0 | 0 | 2 | 4.506 | 19.70 |
| -1 | 0 | 2 | 4.343 | 20.45. |
| 1 | 0 | 2 | 4.333 | 20.50 |
| 3 | 1 | 0 | 4.251 | 20.90 |
| 4 | 0 | 0 | 4.014 | 22.14 |
| -2 | 0 | 2 | 3.936 | 22.59 |
| 2 | 0 | 2 | 3.922 | 22.67 |
| -3 | 1 | 1 | 3.850 | 23.11 |
| 3 | 1 | 1 | 3.840 | 23.17 |
| 0 | 1 | 2 | 3.788 | 23.49 |
| -1 | 1 | 2 | 3.690 | 24.12 |
| 1 | 1 | 2 | 3.684 | 24.16 |
| -4 | 0 | 1 | 3.673 | 24.23 |

### Form B

Form B can be used as an intermediate for the preparation of form A.

Form B is a di-hydrated form that crystallizes in the triclinic system (space group P1, Z=1), with unit cell parameters at T=295 K as follows:

| | |
|---|---|
| a = 8.856(2) Å, | α = 100.76(1)° |
| b = 18.482(1) Å, | β = 90.06(1)° |
| c = 6.904(2) Å | γ = 78.35(1)° |
| | V = 1086.5 Å³ |
| | density = 1.198 |

The asymmetric unit is composed of two molecules of 3-beta-amino-17-methylene-androstane-6-alpha,7-beta-diol hydrochloride and 4 molecules of water.

As all lines present on the diffraction diagram are indexed, form B, as obtained according to the crystallization process described below in Example 3, is a pure physical form.

The indexing of the initial 30 lines of the powder X-ray diffraction pattern diagram of form B of 3-beta-amino-17-methylene-androstane-6-alpha,7-beta-diol hydrochloride at T=295 K, in lattice spacing and in “mean λ_{Cu Ka}” 2θ positions, gives the following result:

| | | | | 2-theta |
|---|---|---|---|---|
| | | | Lattice | “mean λCu K+” |
| h | k | l | spacing (Å) | 1.54184 Å |
| 0 | 1 | 0 | 17.770 | 4.97 |
| 0 | 2 | 0 | 8.885 | 9.96 |
| 1 | 0 | 0 | 8.667 | 10.21 |
| 1 | 1 | 0 | 8.509 | 10.40 |
| -1 | 1 | 0 | 7.227 | 12.25 |
| 1 | 2 | 0 | 6.960 | 12.72 |
| 0 | 0 | 1 | 6.778 | 13.06 |
| 0 | -1 | 1 | 6.777 | 13.06 |
| 0 | 1 | 1 | 5.966 | 14.85 |
| 0 | -2 | 1 | 5.964 | 14.85 |
| 0 | 3 | 0 | 5.923 | 14.96 |
| -1 | 2 | 0 | 5.651 | 15.68 |
| -1 | -1 | 1 | 5.446 | 16.28 |
| 1 | 0 | 1 | 5.441 | 16.29 |
| 1 | 3 | 0 | 5.438 | 16.30 |
| -1 | 0 | 1 | 5.243 | 16.91 |
| 1 | -1 | 1 | 5.238 | 16.93 |
| -1 | -2 | 1 | 5.172 | 17.15 |
| 1 | 1 | 1 | 5.168 | 17.16 |
| 0 | 2 | 1 | 4.953 | 17.91 |
| 0 | -3 | 1 | 4.952 | 17.91 |
| -1 | 1 | 1 | 4.695 | 18.90 |
| 1 | -2 | 1 | 4.690 | 18.92 |
| -1 | -3 | 1 | 4.594 | 19.32 |
| 1 | 2 | 1 | 4.591 | 19.33 |
| -1 | 3 | 0 | 4.481 | 19.82 |
| 0 | 4 | 0 | 4.443 | 19.99 |
| 2 | 1 | 0 | 4.425 | 20.07 |
| 2 | 0 | 0 | 4.334 | 20.49 |
| 1 | 4 | 0 | 4.331 | 20.51 |

Form C

Form C is a mono-hydrate form that crystallizes in the triclinic system (space group Pl, Z=1), with the unit cell parameters at T=295 K as follows:

| | |
|---|---|
| a = 7.2328(5) Å, | α = 97.135(6)° |
| b = 21.063(2) Å, | β = 102.653(5)° |
| c = 7.1563(5) Å | γ = 91.177(6)° |
| | V = 1054.2 Å³ |
| | density = 1.I78 |

The asymmetric unit is composed of two molecules of 3-beta-amino-17-methylene-androstane-6-alpha,7-beta-diol hydrochloride and 2 molecules of water.

As all lines present on the diffraction diagram are indexed, the C form, as obtained according to the process of crystallization described below in Example 4, is a pure physical form.

The indexing of the initial 30 lines of the powder X-ray diffraction pattern diagram of form B of 3-beta-amino-17-methylene-androstane-6-alpha,7-beta-diol hydrochloride at T=295 K, in lattice spacing and “mean λ_{Cu Ka}”2θ positions “λ_{Cu Ka}mid”, gives the following result:

| | | | | 2 theta |
|---|---|---|---|---|
| | | | Lattice | “mean λCu Kα” |
| h | k | l | Spacing (Å) | 1.54184 Å |
| 0 | 1 | 0 | 20.875 | 4.23 |
| 0 | 2 | 0 | 10.437 | 8.47 |
| 1 | 0 | 0 | 7.049 | 12.56 |
| 0 | 3 | 0 | 6.958 | 12.72 |
| 0 | 0 | 1 | 6.922 | 12.79 |
| 0 | -1 | 1 | 6.845 | 12.93 |
| -1 | 1 | 0 | 6.780 | 13.06 |
| 1 | 1 | 0 | 6.581 | 13.46 |
| 0 | 1 | 1 | 6.325 | 14.00 |
| 0 | -2 | 1 | 6.155 | 14.39 |
| -1 | 2 | 0 | 5.980 | 14.81 |
| 1 | 2 | 0 | 5.712 | 15.51 |
| -1 | 0 | 1 | 5.604 | 15.81 |
| -1 | -1 | 1 | 5.506 | 16.10 |
| 0 | 2 | 1 | 5.447 | 16.27 |
| -1 | 1 | 1 | 5.323 | 16.66 |
| 0 | -3 | 1 | 5.267 | 16.83 |
| 0 | 4 | 0 | 5.219 | 16.99 |
| -1 | -2 | 1 | 5.083 | 17.45 |
| -1 | 3 | 0 | 5.079 | 17.46 |
| 1 | 3 | 0 | 4.834 | 18.35 |
| -1 | 2 | 1 | 4.804 | 18.47 |
| 0 | 3 | 1 | 4.612 | 19.24 |
| -1 | -3 | 1 | 4.516 | 19.66 |
| 1 | -1 | 1 | 4.474 | 19.84 |
| 1 | 0 | 1 | 4.465 | 19.88 |
| -0 | -4 | 1 | 4.459 | 19.91 |
| -1 | 4 | 0 | 4.297 | 20.67 |
| 1 | -2 | 1 | 4.290 | 20.71 |
| 1 | 1 | 1 | 4.266 | 20.82 |

The invention is directed to the crystalline forms A, B or C as previously described for use as a medicament.

The crystalline forms A, B or C of 3-beta-amino-17-methylene-androstane-6-alpha,7-beta-diol hydrochloride can be used orally, parenterally, topically by inhalation, or via implants. They can be prescribed as plain or sugar-coated tablets, capsules, granules, suppositories, pessaries, injectable preparations, ointments, creams, gels, microspheres, implants, or patches, all of which may be prepared according to usual methods.

The crystalline forms A, B or C of 3-beta-amino-17-methylene-androstane-6-alpha,7-beta-diol hydrochloride can be mixed with excipients, diluents and all vehicles known to a person skilled in the art for the manufacture of pharmaceutical compositions. As examples of excipients usually employed in these pharmaceutical compositions the following can be cited: talc, acacia gum, lactose, starch, magnesium stearate, cacao butter, aqueous or non-aqueous vehicles, fatty substances of animal or vegetal origin, paraffin derivatives, glycols, and various wetting, dispersing or emulsifying agents and preservatives.

The invention extends to pharmaceutical compositions containing as active ingredient at least one of the crystalline forms A, B or C of 3-beta-amino-17-methylene-androstane-6-alpha,7-beta-diol hydrochloride as defined above and one or more pharmaceutically acceptable excipients, diluents or supports.

The invention is also directed to the use of the crystalline forms A, B or C of 3-beta-amino-17-methylene-androstane-6-alpha,7-beta-diol hydrochloride as definite above for the preparation of a medicament for the treatment of inflammatory diseases, such as asthma.

The following examples illustrate the invention without however limiting it.

### EXAMPLE 1

3-beta-amino-17-methylene-androstane-6-alpha,7-beta-diol hydrochloride, Form A.

250 mg of compound of formula I is dissolved at ambient temperature in minimum amount of methanol. Isopropylic ether is added until the onset of precipitation. After filtration, 195 mg of form A of is obtained.

### EXAMPLE 2

3-beta-amino-17-methylene-androstane-6-alpha,7-beta-diol hydrochloride, Form A.

250 mg of compound of formula I is dissolved at ambient temperature in minimum amount of ethanol. Water is added until the onset of crystallization; polymorph B of is obtained.

Then after evaporation under a stream of nitrogen at ambient temperature, form A is obtained.

### EXAMPLE 3

3-beta-amino-17-methylene-androstane-6-alpha,7-beta-diol hydrochloride, Form B.,

Left for 3 days under a relative humidity above 95% form A of converts to form B

### EXAMPLE 4

3-beta-amino-17-methylene-androstane-6-alpha,7-beta-diol hydrochloride, Form C.,

250 mg of compound of formula I is dissolved at ambient temperature in minimum amount of methyl ethyl ketone (MEK). After transfer in water by azeotropic distillation at constant volume and equilibration under a relative humidity above 97%, form C of compound of formula lis obtained.

1) Crystalline form A of 3-beta-amino-17-methylene-androstane-6-alpha,7-beta-diol hydrochloride corresponding to the structure: 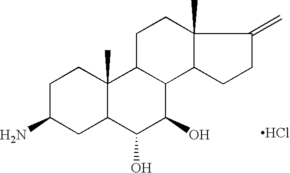
characterized by the fact that the indexing of the first 30 lines of the powder X-ray diffraction pattern diagram at 295 K is:

| | | | | 2 theta |
|---|---|---|---|---|
| | | | lattice spacing | “mean λCu Kα” |
| h | k | l | (Å) | 1.54184 Å |
| 1 | 0 | 0 | 16.058 | 5.50 |
| 0 | 0 | 1 | 9.011 | 9.82 |
| 2 | 0 | 0 | 8.029 | 11.02 |
| −1 | 0 | 1 | 7.872 | 11.24 |
| 1 | 0 | 1 | 7.844 | 11.28 |
| 1 | 1 | 0 | 6.413 | 13.81 |
| −2 | 0 | 1 | 6.007 | 14.75 |
| 2 | 0 | 1 | 5.982 | 14.81 |
| 0 | 1 | 1 | 5.526 | 16.04 |
| 3 | 0 | 0 | 5.353 | 16.56 |
| 2 | 1 | 0 | 5.274 | 16.81 |
| −I | 1 | 1 | 5.229 | 16.96 |
| 1 | 1 | 1 | 5.221 | 16.98 |
| −3 | 0 | 1 | 4.610 | 19.25 |
| 3 | 0 | 1 | 4.594 | 19.32 |
| −2 | 1 | 1 | 4.557 | 19.48 |
| 2 | 1 | 1 | 4.546 | 19.53 |
| 0 | 0 | 2 | 4.506 | 19.70 |
| −1 | 0 | 2 | 4.343 | 20.45 |
| 1 | 0 | 2 | 4.333 | 20.50 |
| 3 | 1 | 0 | 4.251 | 20.90 |
| 4 | 0 | 0 | 4.014 | 22.14 |
| −2 | 0 | 2 | 3.936 | 22.59 |
| 2 | 0 | 2 | 3.922 | 22.67 |
| −3 | 1 | 1 | 3.850 | 23.11 |
| 3 | 1 | 1 | 3.840 | 23.17 |
| 0 | 1 | 2 | 3.788 | 23.49 |
| −1 | 1 | 2 | 3.690 | 24.12 |
| 1 | 1 | 2 | 3.684 | 24.16 |
| −4 | 0 | 1 | 3.673 | 24.23 |

2) Crystalline form A of 3-beta-amino-17-methylene-androstane-6-alpha,7-beta-diol hydrochloride characterized by the fact that the unit cell is monoclinic (space group P2, Z=2) and the unit cell parameters at T=295 K are:

| | |
|---|---|
| a = 16.058(2) Å, | β = 90.24(2)° |
| b = 6.995(1) Å, | V = 1012.2 Å³ |
| c = 9.011(2) Å | density = 1.168 |

3) Di-hydrated crystalline form B of 3-beta-amino-17-methylene-androstane-6-alpha,7-beta-diol hydrochloride, characterized by the fact that the indexing of the lines of the powder X-ray diffraction pattern diagram at 295 K is:

| | | | | 2 theta |
|---|---|---|---|---|
| | | | Lattice spacing | “mean λCu Kα” |
| h | k | l | (Å) | 1.54184 Å |
| 0 | 1 | 0 | 17.770 | 4.97 |
| 0 | 2 | 0 | 8.885 | 9.96 |
| 1 | 0 | 0 | 8.667 | 10.21 |
| 1 | 1 | 1 | 8.509 | 10.40 |
| −1 | 1 | 0 | 7.227 | 12.25 |
| 1 | 2 | 0 | 6.960 | 12.72 |
| 0 | 0 | 1 | 6.778 | 13.06 |
| 0 | −1 | 1 | 6.777 | 13.06 |
| 0 | 1 | 1 | 5.966 | 14.85 |
| 0 | −2 | 1 | 5.964 | 14.85 |
| 0 | 3 | 0 | 5.923 | 14.96 |
| −1 | 2 | 0 | 5.651 | 15.68 |
| −1 | −1 | 1 | 5.446 | 16.28 |
| 1 | 0 | 1 | 5.441 | 16.29 |
| I | 3 | 0 | 5.438 | 16.30 |
| 1 | 0 | 1 | 5.243 | 16.91 |
| 1 | −1 | 1 | 5.238 | 16.93 |
| −1 | −2 | 1 | 5.172 | 17.15 |
| 1 | 1 | 1 | 5.168 | 17.16 |
| 0 | 2 | 1 | 4.953 | 17.91 |
| 0 | −3 | 1 | 4.952 | 17.91 |
| −1 | 1 | 1 | 4.695 | 18.90 |
| 1 | −2 | 1 | 4.690 | 18.92 |
| −1 | −3 | 1 | 4.594 | 19.32 |
| 1 | 2 | 1 | 4.591 | 19.33 |
| −1 | 3 | 0 | 4.481 | 19.82 |
| 0 | 4 | 0 | 4.443 | 19.99 |
| 2 | 1 | 0 | 4.425 | 20.07 |
| 2 | 0 | 0 | 4.334 | 20.49 |
| 1 | 4 | 0 | 4.331 | 20.51 |

4) Di-hydrated crystalline form B of 3-beta-amino-17-methylene-androstane-6-alpha,7-beta-diol hydrochloride, characterized by the fact that the unit cell is triclinic (space group P1, Z=1) and the unit cell parameters at T=295 are:

| | |
|---|---|
| a = 8.85 6(2) Å, | α = 100.76(1)° |
| b = 18.482(1) Å, | β = 90.06(1)° |
| c = 6.904(2) Å | γ = 78.35(I) |
| | V = do 1086.5 Å³ |
| | density = 1.198 |

5) Monohydrate crystalline form C of 3-beta-amino-17-methylene-androstane-6-alpha,7-beta-diol hydrochloride, characterized by the fact that the indexing of the first 30 lines of the powder X-ray diffraction pattern diagrams at 295 K is

| | | | | 2 theta |
|---|---|---|---|---|
| | | | Lattice spacing | “mean λCu Kα” |
| h | k | l | (Å) | 1.54184 Å |
| 0 | 1 | 0 | 20.875 | 4.23 |
| 0 | 2 | 0 | 10.437 | 8.47 |
| 1 | 0 | 0 | 7.049 | 12.56 |
| 0 | 3 | 0 | 6.958 | 12.72 |
| 0 | 0 | 1 | 6.922 | 12.79 |
| 0 | −1 | 1 | 6.845 | 12.93 |
| −1 | 1 | 0 | 6.780 | 13.06 |
| 1 | 1 | 0 | 6.581 | 13.46 |
| 0 | 1 | 1 | 6.325 | 14.00 |
| 0 | −2 | 1 | 6.155 | 14.39 |
| −1 | 2 | 0 | 5.980 | 14.81 |
| 1 | 2 | 0 | 5.712 | 15.51 |
| −1 | 0 | 1 | 5.604 | 15.81 |
| −1 | −1 | 1 | 5.506 | 16.10 |
| 0 | 2 | 1 | 5.447 | 16.27 |
| −1 | 1 | 1 | 5.323 | 16.66 |
| 0 | −3 | 1 | 5.267 | 16.83 |
| 0 | 4 | 0 | 5.219 | 16.99 |
| −1 | −2 | 1 | 5.083 | 17.45 |
| −1 | 3 | 0 | 5.079 | 17.46 |
| 1 | 3 | 0 | 4.834 | 18.35 |
| −1 | 2 | 1 | 4.804 | 18.47 |
| 0 | 3 | 1 | 4.612 | 19.24 |
| −1 | −3 | 1 | 4.516 | 19.66 |
| 1 | −1 | 1 | 4.474 | 19.84 |
| 1 | 0 | 1 | 4.465 | 19.88 |
| 0 | −4 | 1 | 4.459 | 19.91 |
| −1 | 4 | 0 | 4.297 | 20.67 |
| 1 | −2 | 1 | 4.290 | 20.71 |
| 1 | 1 | 1 | 4.266 | 20.82 |

6) Monohydrate crystalline form C of 3-beta-amino-17-methylene-androstane-6-alpha,7-beta-diol hydrochloride, characterized by the fact that the unit cell is triclinic (space group P1, Z=1) and the unit cell parameters T=295 K are:

| | |
|---|---|
| a = 7.2328(5) Å, | α = 97.135(6)° |
| b = 21.063(2) Å, | β = 102.653(5)° |
| c = 7.1563(5) Å | γ = 91.177(6)° |
| | V = 1054.2 Å³ |
| | density = 1.178 |

7) A process for the preparation of form A as defined in claims 1 or 2, characterized by the fact that crystallization takes place in a mixture of alcohol and ether and particularly in an isopropylic methanol-ether mixture.

8) A process for the preparation of form C as defined in claim 5 or 6, characterized by the fact that 250 mg of compound of formula (1) are dissolved at ambient temperature in a solvent such as methyl ethyl ketone (MEK); and then transferred in water by azeotropic distillation at constant volume and equilibration at a relative humidity above 97%.

9) As medications, crystalline forms A, B or C as defined by claims 1 to 6.

10) A pharmaceutical composition characterized by the fact that it comprises form A of 3-beta-amino-17-methylene-androstane-6-alpha,7-beta-diol hydrochloride in a pure state or possibly in combination with either one of/or both crystalline forms B or C and/or in combination with any compatible and pharmaceutically acceptable excipient or inert diluent.

11) A method of treating inflammatory diseases, wherein the method comprises administering a pharmaceutical composition comprising one or more cystalline forms A, B or C as defined by claims 1 to 6 to a human.
